(19) United States
(12) Patent Application Publication
(10) Pub. No.: US 2024/0382333 A1
(43) Pub. Date: Nov. 21, 2024

(54) JOINT ALIGNMENT DEVICE FOR IMPROVED MOBILITY AND FUNCTION

(71) Applicant: Henry Lu, Seattle, WA (US)

(72) Inventor: Henry Lu, Seattle, WA (US)

(21) Appl. No.: 18/627,496

(22) Filed: Apr. 5, 2024

Related U.S. Application Data
(60) Provisional application No. 63/467,141, filed on May 17, 2023.

(51) Int. Cl. A61F 5/01

(52) U.S. Cl. CPC A61F 5/0123; A61F 5/0127; A61F 5/013; A61F 2005/0158

(57) ABSTRACT

The present disclosure describes a multi-step joint facilitation and locking system that allows pressure to be applied in a spiral pattern to joints with an asymmetrical pattern. The system includes a primary and secondary locking mechanism, elastic straps, and a compression sleeve with a silicone-like layer. The system can be used on various joints such as the knee, ankle, and elbow to facilitate accessory joint motion and promote proper joint alignment. The invention provides a way to fine-tune the amount of pressure applied to the joint and to apply pressure in a specific direction to restore natural joint position.

### CROSS-REFERENCE TO RELATED PATENT DOCUMENTS

This patent application claims the benefit of priority of U.S. Provisional Application No. 63/467,141, entitled “JOINT ALIGNMENT DEVICE FOR IMPROVED MOBILITY AND FUNCTION,” filed 17 May 2023, which is hereby incorporated herein by reference in its entirety.

### TECHNICAL FIELD

The present invention relates to the field of medical devices, specifically focusing on the development of an alignment apparatus aimed at enhancing joint functionality and mobility. This innovative joint alignment device is designed to mitigate joint pain and address issues of limited joint mobility by ensuring correct positioning and alignment of joints. By emphasizing the importance of proper joint alignment, the invention seeks to significantly improve overall joint mobility and function, offering a novel solution for individuals experiencing discomfort or reduced mobility due to misalignment. This advancement not only contributes to the field of orthopedics and physical therapy but also opens new pathways for non-invasive interventions aimed at enhancing the quality of life for patients suffering from joint-related ailments.

### BACKGROUND

The human body's joints are complex structures that allow for movement and range of motion. However, when joints are misaligned, the result can be joint pain and limited mobility, which can greatly reduce an individual's quality of life. Several factors contribute to joint pain and limited mobility, including improper joint alignment and the loss of accessory joint motions. Current solutions to address joint pain and limited mobility often involve the use of medication or surgery, both of which can be costly and carry potential risks. Moreover, these solutions may not address the underlying causes of the problem, which can result in recurring joint pain and mobility issues. Over time, repetitive movements and overuse can cause joints to become misaligned, leading to joint pain, decreased range of motion, decreased strength, and limited functional mobility, especially in weight-bearing joints. The invention seeks to address this issue by promoting proper joint alignment and allowing the joint to move closer to its natural joint motions. This, in turn, allows for an increase in joint mobility, strength, and function.

The previous technologies have focused on stabilizing the joint by limiting motion at the joint line and controlling both aspects of the joint. For example, knee braces have focused on creating devices at both sides of the joint line to apply forces or stabilizing straps or supports to counter joint misalignment. However, these devices have a limited ability to control the tri-planar motion of the joint and do not consider the inherent asymmetries found in each joint. The previous devices have also attempted to reduce tension at the musculotendon's junction by placing pressure on or around the joint line and tendon to change the fulcrum of the joint. However, it is difficult to overcome these forces, and it may require sacrificing function and mobility for stability. To address these problems, a new solution has been developed that promotes proper joint alignment and improves overall joint mobility and position. In summary, this invention represents a significant improvement over existing solutions for joint pain and limited mobility, by addressing the underlying causes of the problem and providing a safe, effective, and non-invasive solution. It is hoped that this invention will help improve the quality of life for individuals suffering from joint pain and limited mobility and reduce the need for more invasive and costly treatments.

### SUMMARY

The present invention discloses a joint alignment device for improved mobility and function. The invention is about addressing joint pain and limited joint mobility by promoting proper joint alignment to improve overall joint mobility and position. It seeks to facilitate proper joint alignment by providing a guiding force that allows the joint to move closer to its natural joint motions, which in turn increases joint mobility, strength, and function. Unlike previous technologies that focus on stabilizing the joint by limiting motion at the joint line, this invention considers the asymmetries found in each joint, which give each bone in the joint an advantage for a particular movement in the joint motion. By doing so, it can assist in controlling the tri-planar motion of the joint and improve joint mobility and function without sacrificing stability or limiting natural movement.

The disclosed solution has been developed that promotes proper joint alignment and improves overall joint mobility and position. The present invention accomplishes this by providing a guiding force that allows the joint to move closer to its natural joint motions. By doing so, it facilitates proper joint alignment and reduces the strain and stress on the joint structures. The invention is based on the understanding that all joint motions require a combination of movements in three planes, which are also known as tri-planar joint motion. Each joint has its own specific combination of each motion at each plane. For example, the knee joint may have primary motions in the sagittal plane for flexion and extension, but it also contains movements in the frontal and transverse planes. Moreover, each joint also contains accessory joint motions that assist the primary motion and allow for increased power generation and shock absorption.

The present invention proposes a new approach that addresses these limitations by promoting proper joint alignment to improve joint mobility and position. The invention provides a guiding force that allows the joint to move closer to its natural joint motions, facilitating proper joint alignment and increasing joint mobility, strength, and function. Unlike previous devices, this invention considers the natural asymmetries of the body and uses this information to stabilize the joint using unequal forces on each side of the joint. By doing so, the invention can increase stability without limiting the natural movement and function of the joint. The disclosed device has been designed to promote proper joint alignment and mobility by applying a spiral force around one side of the joint line, specifically the concave aspect of the knee joint via the tibial bone. Unlike previous technologies that try to control both aspects of the joint, this device takes advantage of the natural asymmetries found in each joint by providing a guiding force to allow the joint to move closer to its natural intended motion, thereby optimizing joint motion and improving function. By using the natural structure of the tibial bone, the device restores asymmetrical movement in the tibial and reduces excessive amounts of tibial external rotation, which allows for an increase in tibial medial rotation and promotes proper joint alignment. The device facilitates and supports proper joint movement via a tri-planar force without being too bulky or restricting joint movement. Additionally, the device restores the accessory motions that are not controlled by a muscle, allowing the structural engineering of the joint to be optimized. Overall, this invention aims to improve joint mobility, reduce pain, and improve function by promoting proper joint alignment through the application of a spiral force.

The present invention discloses a joint alignment device designed to aid in the stabilization, alignment, and functional improvement of joints experiencing discomfort or mobility issues. This device integrates several key components and features that work in synergy to provide targeted support and pressure to the joint, enhancing both mobility and comfort for the user. The device comprises two main elements, the primary and secondary straps, both crafted from an elastic material that allows for flexibility and a snug fit around the joint. These straps are unique in that one side is coated with silicone, which is intended to face the user's skin. The silicone side plays a crucial role in preventing the device from slipping during movement, ensuring that the applied pressure remains consistent and effective. Further, an anchor point is strategically placed on the secondary strap, serving as a dedicated site for attaching it to the primary strap. This connection point is crucial for maintaining the structural integrity of the device, ensuring that the straps work together cohesively to provide the necessary support to the joint. The primary strap is equipped with a two-stage locking mechanism, located on its silicone side. This mechanism includes a first stage lock and a second stage lock, both of which are crucial for securing the device in place around the joint. The locking mechanism is adjustable, allowing users to customize the fit and level of support according to their specific needs. The locking strap is an extension of the primary and secondary straps, designed to encircle a sleeve or sock that is worn over the joint. This sleeve or sock provides additional compression, enhancing the stability and support offered by the device. The compression is mild to moderate, balancing support with comfort to prevent any restriction of circulation. One of the most distinctive features of this device is its ability to apply pressure in a spiral pattern around the joint. This design is intended to mimic the natural movement patterns of the joint, thereby promoting proper alignment, and improving joint function. The spiral pressure application is particularly effective for joints with asymmetrical motion patterns, such as the knee, ankle, elbow, and wrist. To facilitate case of use and adjustability, the non-silicone side of the straps is fitted with a Velcro hook. This hook interacts with a corresponding Velcro loop to attach the straps together securely. This feature allows for easy adjustment of the device's tension and compression around the joint, enabling users to find the most comfortable and effective fit for their individual condition.

The joint alignment device features a nuanced and user-centric design, incorporating a dual-stage locking mechanism that is pivotal for its functionality and adaptability. This system comprises a first stage lock and a second stage lock, both of which are secured using Velcro material-a choice that significantly enhances the device's usability. The Velcro material, renowned for its reliability and ease of use, allows users to adjust the tension and compression applied by the device with remarkable precision. The first stage lock, strategically positioned at the center of the primary strap, provides the initial secure fit around the joint, ensuring the device is snugly in place. Following this, the second stage lock, located at one end of the primary strap and acting as a joint facilitation tail, allows for the application of additional pressure. This can be adjusted in a spiral pattern around the joint, enabling a distribution of force that can be finely tuned to the user's specific needs. The inherent adjustability offered by the Velcro-based locking mechanism thus empowers users to customize the fit and level of support provided by the device, tailoring it to their unique requirements for optimal joint support and mobility enhancement.

The joint alignment device has been engineered to include a robust feature that significantly enhances its durability and effectiveness: a cross-pattern stitching at the anchor point. This meticulous design detail is crucial for reinforcing the connection between the primary and secondary straps, ensuring they remain securely attached even under the stress of dynamic and prolonged use. The cross-pattern stitching, characterized by intersecting lines of thread that form a sturdy lattice at the anchor point, not only increases the tensile strength of the attachment but also distributes stress more evenly across the joint. This prevents focal points of strain that could lead to wear or detachment over time, thereby maintaining the integrity of the device's structure. As a result, users can rely on a consistent level of support and alignment correction from the device, enhancing its overall efficacy in promoting joint health and function.

The joint alignment device has been designed with versatility and specificity in mind, offering a range of wearable options to cater to different joint support needs across the body. This range includes a leg sleeve, forearm sleeve, and a traditional sock, each carefully crafted to provide targeted support to specific joints such as the knee, ankle, elbow, and wrist. This selection ensures that users can choose the most appropriate and comfortable form of support for their particular condition or area of discomfort. Whether it's stabilizing the knee during recovery from an injury, supporting the ankle in athletic endeavors, reducing strain on the elbow during repetitive movements, or alleviating wrist discomfort from prolonged computer use, the device offers a tailored solution. By encompassing a variety of joints and allowing for precise application to the affected area, the device maximizes its effectiveness and user satisfaction, making it a versatile tool in the management and support of joint health.

In the design of the joint alignment device, the silicone side of both the primary and secondary straps is enhanced with a textured surface, a feature meticulously engineered to augment the device's functionality and user experience. This textured silicone surface plays a pivotal role in increasing friction between the strap and the user's skin, effectively minimizing any potential for slippage or shifting during various physical activities. Whether the user is engaged in high-intensity sports, moderate exercise, or simply going about their daily routine, the increased grip provided by the textured surface ensures that the device remains securely in place, maintaining optimal alignment and support of the joint without necessitating frequent readjustments. This design consideration not only bolsters the device's efficacy in providing sustained joint support but also boosts wearer confidence, knowing that the device will reliably stay put, allowing them to focus on their activities with enhanced joint protection and comfort.

The joint alignment device further introduces an innovative alternate embodiment with its overlapping locking mechanism, designed to significantly enhance the device's stability and allow for customized pressure distribution across the joint. This advanced feature consists of overlapping sections of the primary and secondary straps, ingeniously integrated with additional Velcro fastening points. These overlapping sections, when secured with Velcro, not only increase the contact area between the straps for a more secure fit but also enable users to precisely adjust the level of compression and support provided by the device. The inclusion of multiple fastening points allows for a granular level of control over how pressure is applied, facilitating a tailored approach to joint support that can be adjusted to suit individual needs and activities. This mechanism is particularly beneficial in situations requiring specific pressure patterns to correct or support joint alignment, offering a dynamic solution that adapts to the user's movement and enhances the overall efficacy of the device in promoting joint health and mobility.

The joint alignment device further employs a meticulously designed primary strap that features a first stage lock strategically positioned at the center between the two ends of the strap. This central placement of the first stage lock is crucial for ensuring an initial secure fit around the joint, acting as the foundational layer of support upon which further adjustments can be made. By situating this lock at the midpoint of the strap, the device achieves a balanced distribution of pressure from the outset, providing a stable base that snugly encircles the joint. This initial positioning is essential for the effective application of the device, as it ensures that the strap is correctly aligned with the joint, offering immediate stabilization and support before any additional tension adjustments are made with the second stage lock or other mechanisms. The central location of the first stage lock thus plays a pivotal role in the overall functionality and efficiency of the device, laying the groundwork for customized support and alignment correction.

The joint alignment device further incorporates a second stage lock on the primary strap, ingeniously positioned at one end of the strap to serve a dual purpose. This second stage lock acts as a joint facilitation tail, a key feature designed to apply additional pressure in a strategic spiral pattern around the joint. By situating this lock at the strap's extremity, it allows for the extension and precise adjustment of the device's tension, enabling users to fine-tune the level of support and compression required for their specific joint issues. The spiral pattern of pressure application is critical for mimicking the natural movements of the joint, thereby promoting proper alignment, and enhancing mobility without restricting movement unduly. This method of applying pressure ensures that support is not only comprehensive but also targeted, addressing the asymmetrical patterns of movement that are often present in joint conditions. The second stage lock, therefore, plays a crucial role in the device's ability to provide customized therapeutic support, making it a vital component in the overall design aimed at optimizing joint function and comfort.

In the design of the joint alignment device, the secondary strap plays a pivotal role in enhancing the functionality and effectiveness of the support provided to the joint. This strap is meticulously attached on top of the primary strap, with a deliberate orientation that places its silicone side facing inward, directly against the non-silicone side of the primary strap. This specific arrangement is critical for several reasons. Firstly, the silicone side of the secondary strap increases the friction between the two straps, preventing any unwanted slipping or shifting that could compromise the stability and precision of the support being applied. Secondly, this configuration facilitates a more nuanced application of pressure and support by leveraging the clastic and non-slip properties of the silicone material. The inward-facing silicone side of the secondary strap works in conjunction with the primary strap to secure the device in place, ensuring that the therapeutic pressure is maintained consistently around the joint. This strategic layering and orientation of the straps significantly enhance joint facilitation, offering a sophisticated mechanism to adjust support levels and optimize joint function and comfort effectively.

The integration of the primary and secondary straps with the sleeve or sock in the joint alignment device is achieved through a methodical sewing process, which plays a crucial role in ensuring the longevity and resilience of the device. This sewing technique ensures that the straps are not only securely affixed to the sleeve or sock but also that the attachment is durable enough to withstand the rigors of daily wear and tear, including frequent movements and adjustments. By opting for sewing as the method of attachment, the design guarantees a permanent bond between the straps and the base garment, thereby eliminating the risk of detachment or loosening that could compromise the device's effectiveness in providing consistent support and compression. This durable attachment is particularly important for a device intended for repeated use, as it ensures that the device remains functional and reliable over time, regardless of the frequency of use or the intensity of physical activity. The sewn attachment thus underscores the device's commitment to quality and durability, offering users a dependable solution for joint alignment and support.

The joint alignment device has been crafted to cater to a wide spectrum of joint-related ailments, extending its therapeutic benefits beyond the conventional scope. It is specifically designed to provide relief and support for conditions such as tennis elbow, golf elbow, wrist injuries, carpal tunnel syndrome, and arthritis. By applying targeted pressure and support to the affected areas, the device effectively addresses the underlying causes of discomfort and immobility associated with these conditions. For tennis and golf elbow, it aids in alleviating the strain on tendons and muscles around the elbow joint. In the case of wrist injuries and carpal tunnel syndrome, it helps by stabilizing the wrist and reducing pressure on the median nerve, thus mitigating pain, and facilitating recovery. For arthritis, the device offers support that can decrease pain and improve joint function. The versatility of the device lies in its ability to adapt its support and compression levels to the specific needs of the user, making it a valuable tool for individuals seeking a non-invasive solution to enhance joint health and regain mobility.

The inclusion of an anchor point on the secondary strap of the joint alignment device represents a critical design feature that significantly enhances its functionality and user experience. This anchor point serves as a dynamic interface for fine-tuning the force exerted by the primary strap onto the joint, offering users the capability to make precise adjustments to the level of support and compression provided. By enabling such meticulous control over the force application, the device caters to the nuanced needs of individual joints, ensuring that the support can be customized to address specific conditions or levels of discomfort. This adjustability is especially beneficial for managing varying degrees of joint issues, allowing for the optimization of joint support based on activity level, recovery stage, or personal comfort preferences. The strategic placement of the anchor point on the secondary strap thus plays a pivotal role in the device's overall effectiveness, facilitating a tailored approach to joint care that enhances mobility, reduces pain, and promotes healing in a targeted and efficient manner.

The joint alignment device further incorporates a Velcro fastening system for both the primary and secondary straps, a design choice that significantly enhances the usability and versatility of the device. This fastening system ensures a secure yet adjustable fit around the joint, catering to the dynamic needs of users across a variety of activities and conditions. The Velcro allows for quick and easy attachment of the straps to the sleeve or sock, facilitating a straightforward application process that users can manage independently. Similarly, the detachability feature offered by the Velcro system means that the device can be easily removed for comfort, cleaning, or adjustment purposes, without compromising the stability and support it provides when in use. This level of convenience and adaptability makes the Velcro fastening system an integral part of the device's design, ensuring that users can maintain an optimal level of joint support while also accommodating their lifestyle and mobility needs with case.

The joint alignment device has been designed to prioritize both hygiene and user convenience, featuring a construction that allows it to be easily removed and washed alongside its accompanying sleeve or sock. This capability ensures that users can regularly clean the device to maintain hygiene and comfort without risking damage to its structural integrity or the efficacy of its locking mechanism. The durability of the attachment points and the resilience of the locking system are preserved through wash cycles, ensuring that the device retains its functionality and support capabilities over time. By facilitating straightforward maintenance, the device addresses a crucial aspect of long-term wearability, allowing users to sustain a clean and effective joint support solution. This design consideration not only extends the lifespan of the device but also enhances user satisfaction by simplifying the care process, ensuring that maintaining the device does not become a burden, thereby encouraging consistent use for optimal joint health.

These and other features and advantages of the present invention will become apparent from the detailed description below, considering the accompanying drawings.

### BRIEF DESCRIPTION OF THE DRAWINGS

Having thus described the invention in general terms, reference will now be made to the accompanying drawings, which are not necessarily drawn to scale, and wherein:

FIG. 1 is a diagram that illustrates a primary strap with a side having no silicone on it, in accordance with various embodiments of the present disclosure.

FIG. 2 is a diagram that illustrates the primary strap with a side having silicone on it, in accordance with various embodiments of the present disclosure.

FIG. 3 is a diagram that illustrates a view of an anchor point and a secondary strap, in accordance with various embodiments of the present disclosure.

FIG. 4 is a diagram that illustrates a view of an anchor point with primary and secondary straps, in accordance with various embodiments of the present disclosure.

FIG. 5 is a diagram that illustrates a view of an anchor point with a cross pattern to attach primary and secondary straps, in accordance with various embodiments of the present disclosure.

FIG. 6 is a diagram that illustrates an attachment of the primary and secondary straps, in accordance with various embodiments of the present disclosure.

FIG. 7 is a diagram that illustrates the silicone side of the straps with a Velcro hook, in accordance with various embodiments of the present disclosure.

FIG. 8 is a diagram that illustrates an attachment of a first-stage lock on the primary strap, in accordance with various embodiments of the present disclosure.

FIGS. 9 and 10 are diagrams that illustrate an attachment of a locking strap to a sleeve, in accordance with various embodiments of the present disclosure.

FIG. 11 is a diagram that illustrates an attachment of the primary and secondary straps with a forearm sleeve, in accordance with various embodiments of the present disclosure.

FIG. 12 is a diagram that illustrates an example of using the straps on a leg, in accordance with various embodiments of the present disclosure.

FIG. 13 is a diagram that illustrates an attachment of the primary and secondary straps with a sock for treating the ankle, in accordance with various embodiments of the present disclosure.

It should be noted that the accompanying figures are intended to present illustrations of exemplary embodiments of the present disclosure. These figures are not intended to limit the scope of the present disclosure. It should also be noted that accompanying figures are not necessarily drawn to scale.

### DETAILED DESCRIPTION

In the following description, for purposes of explanation, numerous specific details are set forth in order to provide a thorough understanding of the present technology. It will be apparent, however, to one skilled in the art that the present technology can be practiced without these specific details. In other instances, structures and devices are shown in block diagram form only in order to avoid obscuring the present technology.

Reference in this specification to “one embodiment” or “an embodiment” means that a particular feature, structure, or characteristic described in connection with the embodiment is included in at least one embodiment of the present technology. The appearance of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Reference will now be made in detail to selected embodiments of the present disclosure in conjunction with accompanying figures. Among various embodiments, the disclosed joint alignment device has been designed to improve mobility and function. The invention is about addressing joint pain and limited joint mobility by promoting proper joint alignment to improve overall joint mobility and position. The embodiments' described figures are intended and provided to illustrate embodiments of the disclosure described below and are not necessarily drawn to scale. In the drawings, like numbers refer to like elements throughout, and the thicknesses and dimensions of some components may be exaggerated for providing better clarity and case of understanding. Further, the terms “a” and “an” herein do not denote a limitation of quantity, but rather denote the presence of at least one of the referenced items.

The disclosed device is an innovative solution aimed at enhancing joint mobility and reducing pain for individuals with asymmetrical motion patterns in their joints. Its unique design involves the application of pressure in a spiral pattern around affected joints, such as the knee, ankle, and elbow, catering specifically to areas that exhibit uneven movement. The core of this device comprises elastic materials strategically wrapped around the bone, notably around the tibia when applied to the knee, to ensure targeted support and alignment. The device incorporates a dual locking mechanism, differentiated into primary and secondary systems, to secure its position and optimize its therapeutic effect. The primary locking mechanism functions in two stages. The first stage is an initial locking system that secures the device in place using a Velcro-like material, ensuring that it remains snug and effective during movement. The second stage, known as the joint facilitation tail, employs a slightly different approach by applying less tension than the initial system and utilizes Velcro-like material for attachment. This tiered approach allows for a base level of support and an additional layer of joint facilitation, promoting better alignment without excessive pressure. Sitting atop the primary mechanism, the secondary locking mechanism serves as an overlay that enhances the device's ability to fine-tune the force exerted on the joint. This feature is crucial for customizing the level of support based on individual needs and for adjusting the pressure as the user's condition improves or changes. By allowing for adjustments, the device ensures that the joint is not only supported but also encouraged to move in a more natural and pain-free pattern. The entire system is integrated into a sleeve or an elastic covering that extends along the length of the targeted bone. This sleeve provides mild to moderate compression, further aiding in the stabilization and support of the joint. The combination of the spiral pressure application, the dual locking mechanism, and the compression sleeve makes this device a comprehensive solution for optimizing joint motion, reducing discomfort, and enhancing overall joint function. It represents a significant step forward in non-invasive joint support technologies, offering a customizable, supportive, and effective treatment option for individuals facing joint mobility challenges.

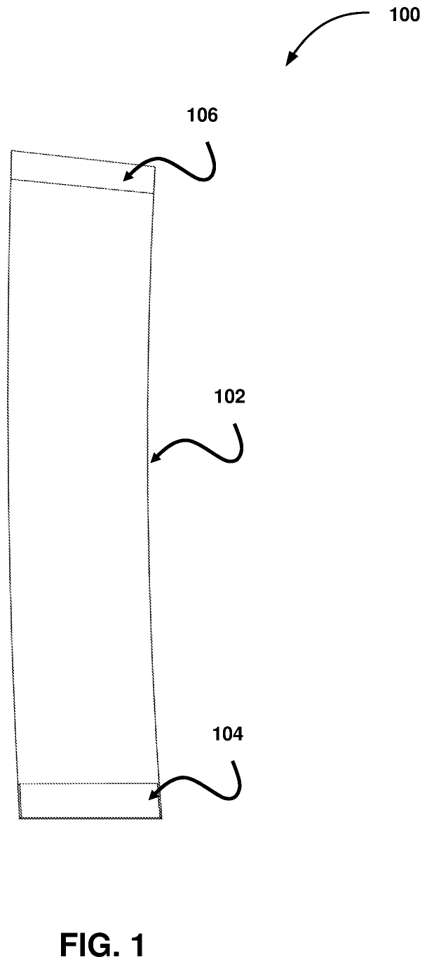

FIG. 1 is a diagram 100 that illustrates a primary strap 102 with a side having no silicone on it, in accordance with various embodiments of the present disclosure. The disclosed device includes the primary strap 102 which has two end portions 104 and 106. The end portions 104 and 106 are primarily used for stitching a secondary strap and Velcro-like materials. Stitching is a method of joining two pieces of fabric or material together using a needle and thread. In this case, one of the end portions of the primary strap 102 is sewn together with the secondary strap and the Velcro-like material to form a locking mechanism. The Velcro-like material is a hook-and-loop fastener system that consists of two components: a hook side and a loop side. The hook side is made up of tiny hooks that attach to the loops on the other side, creating a secure closure. In this case, the Velcro-like material is used to hold the primary and secondary straps in place around the joint.

The device being referred to here is the joint facilitation/locking system. The primary strap 102 is a long elastic strap that can be stretched around a joint, such as the tibia in the case of the knee. The two end portions 104 and 106 of the primary strap 102 are specifically designed for attaching a secondary strap and Velcro. The secondary strap is part of the secondary locking mechanism that allows for further joint facilitation and fine-tuning of the force applied to the joint. The secondary strap is attached to the end portion 104 of the primary strap 102 by stitching them together. The Velcro is used in the two-stage primary locking mechanism of the device. The initial locking system is held in place with a Velcro-like material that is attached to the end portion 106 of the primary strap 102. The joint facilitation tail is not placed on as much tension and is held in place with another Velcro-like material, which is also attached to the end portion 104 of the primary strap 102. The two-stage primary locking mechanism provides a secure and comfortable fit while allowing for joint movement. The primary strap 102 plays a critical role in the joint facilitation/locking system by providing the foundation for the other components of the device to be attached. The two end portions 104 and 106 of the primary strap 102 are specifically designed for attaching the secondary strap and Velcro to enable the device to provide the necessary joint facilitation and locking to optimize joint motion and reduce pain.

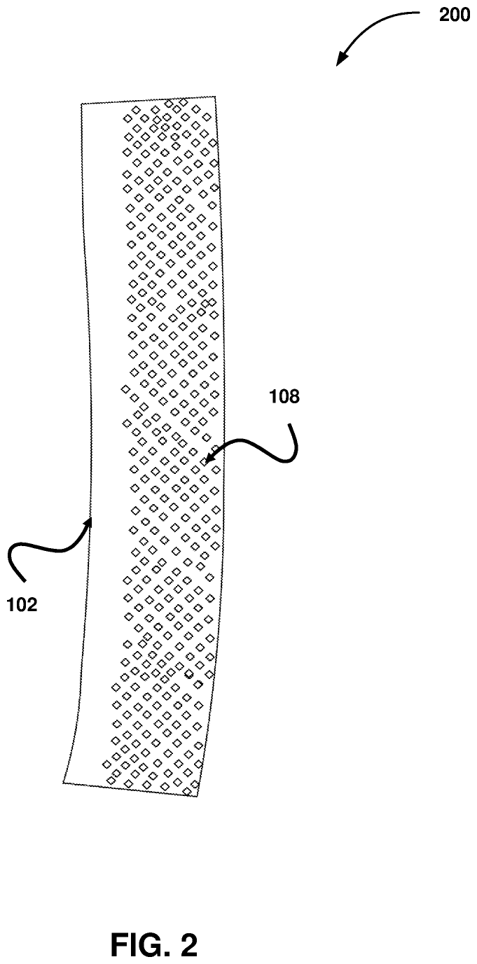

FIG. 2 is a diagram 200 that illustrates the primary strap 102 with a side having silicone 108 on it, in accordance with various embodiments of the present disclosure. This side shown here is opposite to the side shown in FIG. 1. The other side of the primary strap 102 refers to the side opposite to the side that has the end portions 104 and 106. This side of the strap 102 includes a layer of silicone material 108 over it. The purpose of this silicone layer 108 is to prevent the locking mechanism from sliding around on the skin. When the device is applied to the joint, the primary strap 102 with the silicone layer 108 is placed against the skin while the locking mechanism is positioned on top of it. The silicone layer 108 helps to keep the primary strap 102 in place while allowing the locking mechanism to move around and apply the necessary pressure to the joint. This way, the device can be used comfortably and effectively without causing any discomfort or irritation to the skin.

Thus, in FIG. 2, the diagram 200, as described, focuses on the primary strap 102 that incorporates the silicone layer 108. This silicone layer 108 is a crucial element designed to enhance the device's usability and effectiveness when applied to a joint. The silicone, known for its non-slip properties, has been strategically placed on the side of the primary strap 102 that comes into direct contact with the skin. The inclusion of the silicone layer 108 serves a dual purpose. Firstly, it significantly reduces the likelihood of the device moving or sliding across the skin during movement, ensuring that the applied pressure remains consistent and targeted around the joint. This is particularly important for joints that undergo a wide range of motion, such as the knee, ankle, or elbow. Secondly, the silicone 108 provides a gentle grip on the skin, minimizing potential discomfort or irritation, thus allowing the device to be worn for extended periods without causing skin issues. An exemplary case where this design proves beneficial is in the application of the device to an athlete's knee suffering from patellar tracking issues. In such a scenario, the primary strap 102 with the silicone layer 108 is wrapped directly against the skin surrounding the knee. As the athlete moves, the silicone layer 108 prevents the strap 102 from slipping, ensuring that the locking mechanisms stay in position to maintain optimal pressure distribution. This targeted pressure helps correct the patellar movement, reducing pain and enhancing knee function without causing discomfort or skin irritation. The design exemplifies how thoughtful material selection and placement can significantly improve medical device functionality and patient comfort.

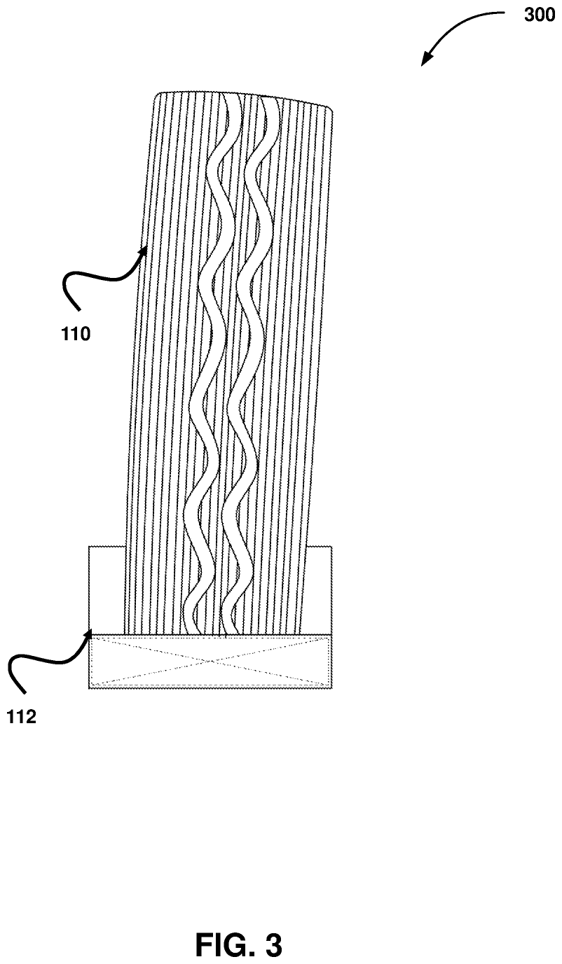

FIG. 3 is a diagram 300 that illustrates a view of an anchor point 112 and a secondary strap 110, in accordance with various embodiments of the present disclosure. The secondary strap 110 is an additional strap that is attached to the primary strap 102 to provide further joint facilitation and allow for fine-tuning of the force applied to the joint. The secondary strap 110 is typically made of an elastic material and can be stretched to apply tension to the joint. The anchor point 112 is a point of attachment for the secondary strap. In the context of this invention, the anchor point 112 is a location on the secondary strap 110 where the primary strap 102 can be anchored or secured in place. The anchor point 112 is typically located near one of the two end portions of the secondary strap 110 and can be adjusted to control the tension applied to the joint by the primary strap 102. By adjusting the anchor point 112, the amount of tension and the direction of the force applied to the joint can be fine-tuned to optimize joint movement and reduce pain. The secondary strap 110 is attached to the primary strap 102.

The secondary strap 110 is an integral part of the device, designed to work in conjunction with the primary strap 102 to enhance the device's ability to facilitate joint movement and adjust the pressure applied to the joint for therapeutic purposes. Made from an clastic material, the secondary strap 110 may be stretched, allowing for the application of variable tension directly to the joint, which is essential for accommodating the unique needs of each user and the specific requirements of different joint conditions. The anchor point 112 serves as a critical interface within the device, providing a secure location on the secondary strap 110 where the primary strap 102 can be effectively anchored or attached. This point 112 is strategically positioned near the ends of the secondary strap 110 to offer adjustability in the tension applied by the primary strap 102, thereby enabling precise control over the force exerted on the joint. By adjusting the position of the anchor point 112, the users or healthcare providers can fine-tune the tension and direction of the force applied, which is pivotal for optimizing joint alignment, enhancing movement, and alleviating pain associated with misalignment or other joint issues. Further, the inclusion of a silicone layer over both the primary and secondary straps 102 and 110 serves an essential function in maintaining the device's position and effectiveness throughout its use. This silicone layer acts as a frictional barrier against the skin and any underlying sleeve, ensuring that the straps 102 and 110 do not slip or shift during movements. This stability is crucial for ensuring the consistent application of the intended tension and pressure to the joint, which is necessary for the therapeutic benefits of the device. The silicone's non-irritating properties also ensure that the device can be worn comfortably for extended periods without risking skin irritation or discomfort, making it a user-friendly solution for ongoing joint support and rehabilitation.

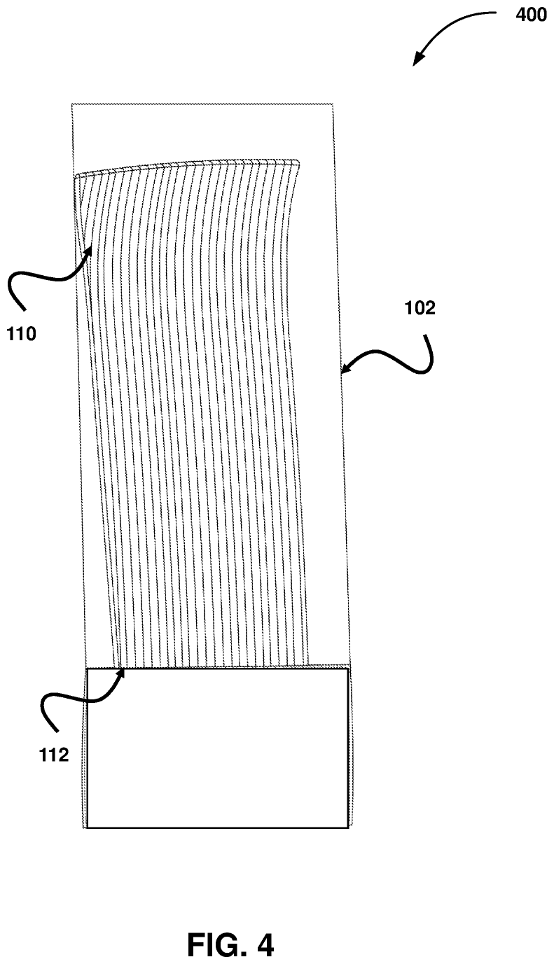

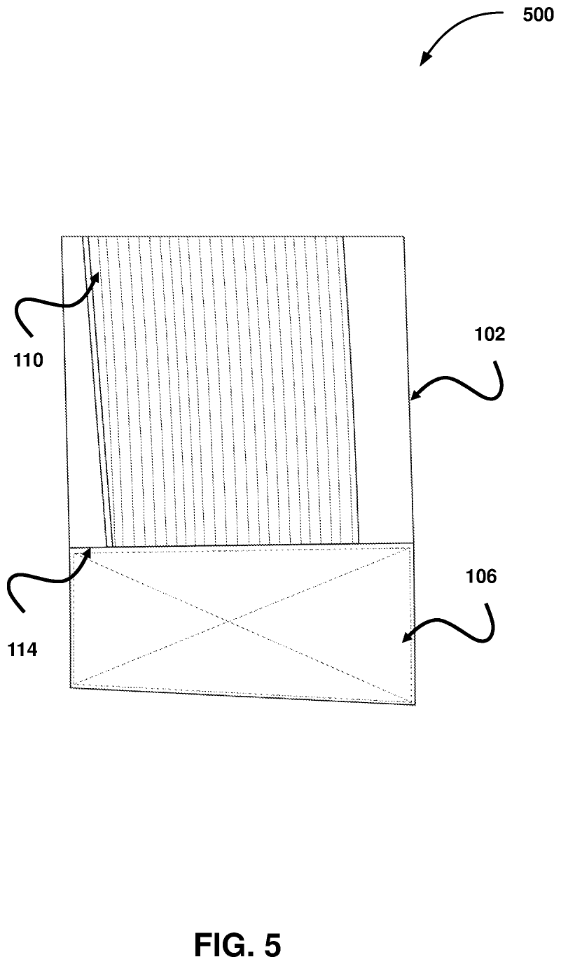

FIG. 4 is a diagram 400 that illustrates a view of an anchor point with primary and secondary straps and FIG. 5 is a diagram 500 that illustrates a view of an anchor point with a cross pattern to attach primary and secondary straps, in accordance with various embodiments of the present disclosure. The device includes the primary strap 102 and the secondary strap 110 that are attached to each other. The attachment is done by sewing and there is an anchor point 112 or 114 on the secondary strap 110 for this purpose. The anchor point is a designated point on the strap that is specifically designed for attaching the strap to another component of the device. In this case, the anchor point 112 or 114 on the secondary strap 110 is used to attach it to the primary strap 102. The attachment is done by sewing the anchor point to the primary strap 102. This creates a secure connection between the two straps, allowing them to work together to provide the intended function of the device. In some embodiments, as shown in FIG. 5, the anchor point 114 with a cross pattern may be used to attach the primary and secondary straps 102 and 110 of the device. The cross pattern refers to the pattern in which the anchor point is designed, where there are two intersecting lines creating a cross shape. This cross pattern provides stability and strength to the attachment point. In some embodiments, the anchor point may be made of a durable material such as plastic or metal, and it is designed to securely hold the primary and secondary straps together. The straps are attached to the anchor point by sewing or other means of attachment such as adhesive. In the context of the invention being discussed, the anchor point 114 with a cross pattern is used to attach the primary and secondary straps 102 and 110 of the device that is applied to joints such as the knee, ankle, or elbow. This attachment point provides stability and allows the device to apply pressure in a spiral pattern, which is crucial to restoring natural movement to the joint.

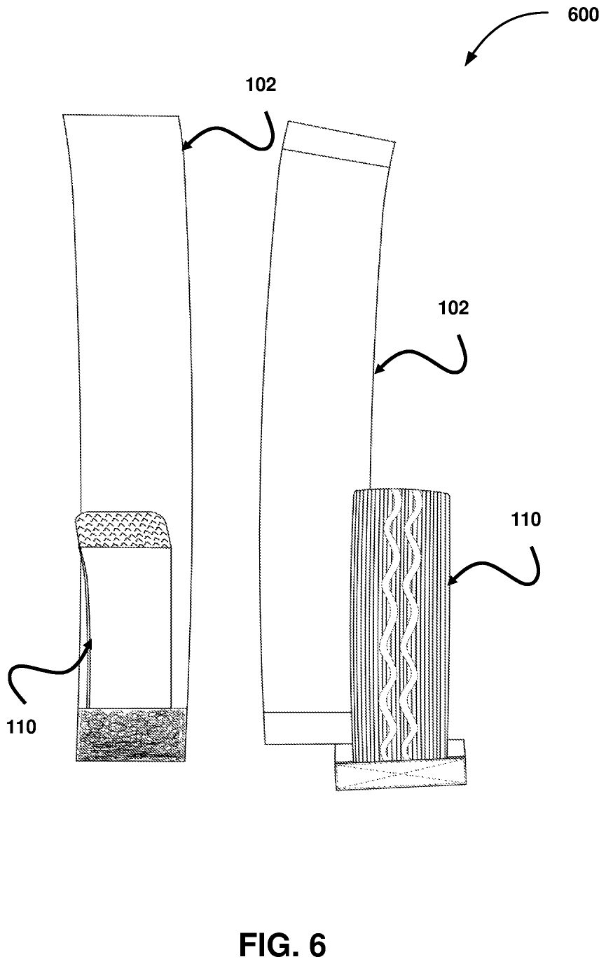

FIG. 6 is a diagram 600 that illustrates the attachment of the primary and secondary straps 102 and 110, in accordance with various embodiments of the present disclosure. The attachment of the primary and secondary straps 102 and 110 is done by sewing them around the anchor point, for example, in a cross pattern as shown in FIG. 5. The primary strap 102 is placed at the bottom with its silicon side facing towards the user's skin, while the secondary strap 110 is placed on top of the primary strap 102 with its non-silicone side facing the secondary strap 110. This way, the silicone side of the secondary strap 110 is not visible and will be touching the non-silicone side of the primary strap 102. The straps are securely attached together through the anchor point, allowing for a stable and adjustable joint facilitation/locking system. There are several possible mechanisms for attaching the primary and secondary straps 102 and 110 of the device, including but not limited to: (1) Hook-and-loop fasteners: this is similar to the current attachment mechanism where one strap has hooks and the other has loops. The two straps are pressed together to form a secure attachment, (2) Snaps: small metal or plastic snaps can be sewn onto the straps and used to attach them together. The snaps would need to be strong enough to hold the straps in place during movement, (3) Buckles: a buckle system could be used to attach the straps together. The buckle would need to be designed to withstand the forces and movement involved in joint motion, and (4) Adhesive: a medical-grade adhesive could be used to attach the straps together. However, this would require careful application and could cause skin irritation for some users. The choice of attaching mechanism would depend on various factors such as case of use, durability, cost, and user comfort. The current hook-and-loop fastener mechanism may be a good balance of these factors, but other mechanisms could be explored for possible improvements.

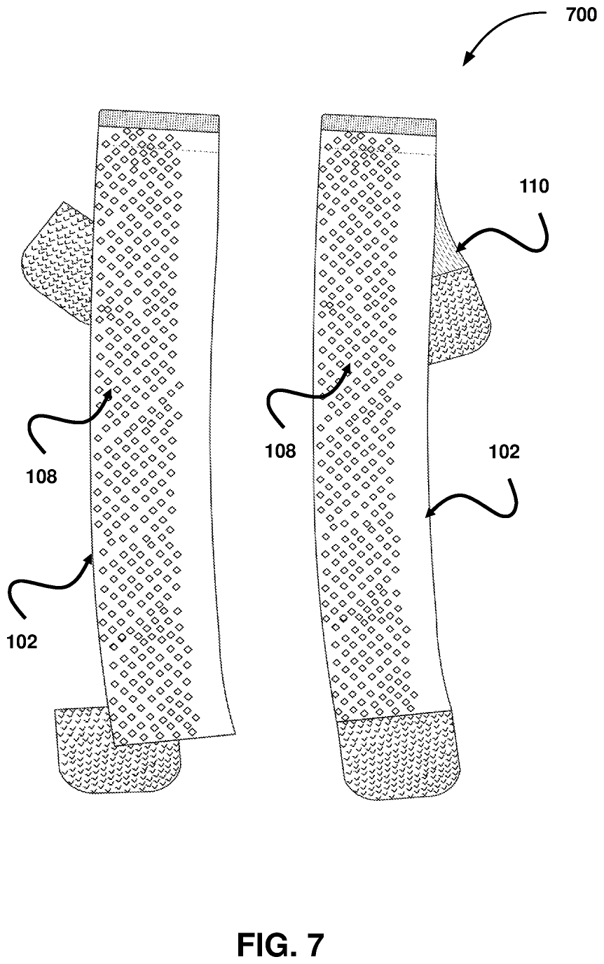

FIG. 7 is a diagram 700 that illustrates the silicone side 108 of the straps 102 and 110 with a Velcro hook. The silicone side 108 of the straps 102 and 110 plays a pivotal role in the device's performance, offering a non-slip surface that ensures the device remains securely in place during various activities. This feature is particularly important for maintaining consistent therapeutic pressure on the joint, a crucial aspect of the device's intended function to improve joint alignment and mobility. The choice of silicone for the side of the straps 102 and 110 that contacts the user's skin is strategic, as silicone is known for its skin-friendly properties, including being hypoallergenic and comfortable for prolonged wear. This consideration is vital for ensuring that users can comfortably use the device for extended periods without experiencing skin irritation or discomfort, which could otherwise hinder compliance with therapeutic regimens. On the opposite side of the straps, the Velcro hook mechanism complements the silicone's functionality by facilitating a secure and adjustable attachment. This design choice allows for a highly customizable fit, enabling users to easily adjust the tightness and positioning of the device around the joint. The Velcro hook engages with a corresponding Velcro loop, enabling a robust yet easily modifiable connection. This adaptability is crucial for accommodating different sizes and shapes of joints, as well as varying degrees of joint swelling or movement, ensuring that the device can be effectively used across a wide range of individuals and conditions. The integration of silicone and Velcro hook-and-loop fasteners into the design of the straps showcases a thoughtful approach to combining materials and mechanisms for optimal performance. This combination ensures that the device not only provides the necessary therapeutic benefits through proper joint alignment and pressure application but also prioritizes user comfort and case of use. By addressing these key aspects, the device stands out as a practical and effective solution for individuals seeking support for joint-related issues, enhancing their ability to maintain active and pain-free lifestyles.

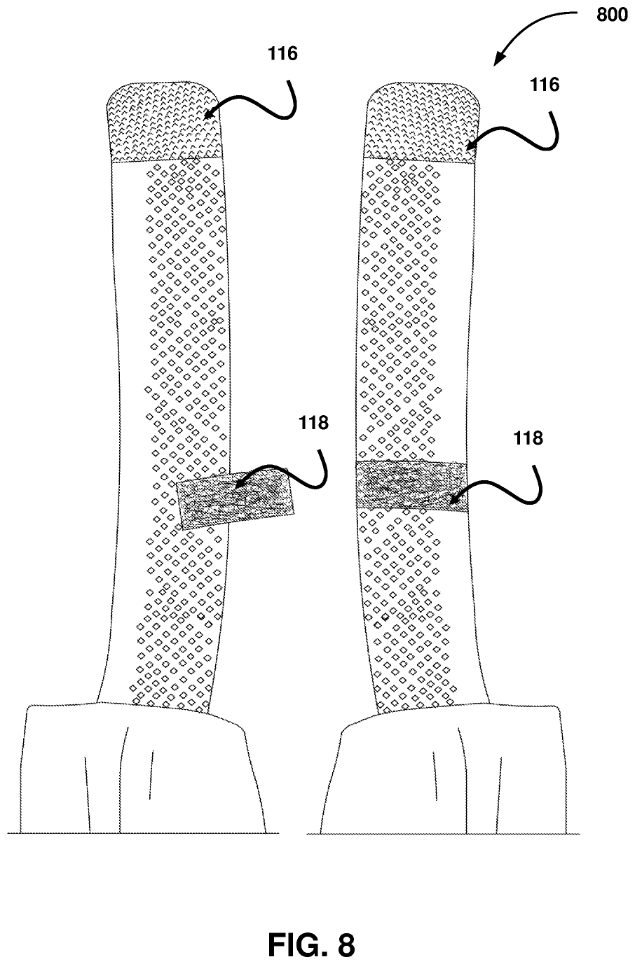

FIG. 8 is a diagram 800 that illustrates an attachment of a first stage lock on the primary strap 102, in accordance with various embodiments of the present disclosure. The diagram 800 introduces a sophisticated dual-stage locking mechanism incorporated into the primary strap 102 of the joint alignment device, showcasing an innovative approach to customizing the application of pressure for joint support and mobility enhancement. This system comprises two distinct Velcro locks: the first stage lock 118 and the second stage lock 116, both strategically positioned on the silicone side of the primary strap to maximize functionality and user comfort. The first stage lock 118 is ingeniously situated between the two ends 104 and 106 of the primary strap 102, ideally in the middle, to serve as the initial point of securement for the device. This lock is attached by sewing the Velcro piece into place, ensuring a durable and reliable connection. The primary function of this first stage lock is to establish the foundational positioning of the device around the joint, providing an initial level of tightness that can be precisely controlled by the user or caregiver. Following the initial setup facilitated by the first stage lock 118, the second stage lock 116 plays a pivotal role in enhancing the device's efficacy. Located at the end 106 of the primary strap 102, and sewn into place, this second lock acts as a joint facilitation tail. Its purpose is to secure the first lock and apply additional pressure as needed, in a controlled spiral pattern around the joint. This method of pressure application is crucial for evenly distributing force across the joint, thereby optimizing joint movement, and potentially reducing pain or discomfort associated with joint misalignments or mobility issues. The two-stage locking mechanism, through its thoughtful design, allows for nuanced adjustments in the force applied to the joint. Users can fine-tune the pressure to their specific needs, facilitating a personalized approach to joint care. This system not only supports the precise application of force in a tri-planar direction, enhancing the therapeutic potential of the device, but also ensures that adjustments can be made easily and efficiently, promoting user independence and comfort in managing their joint health.

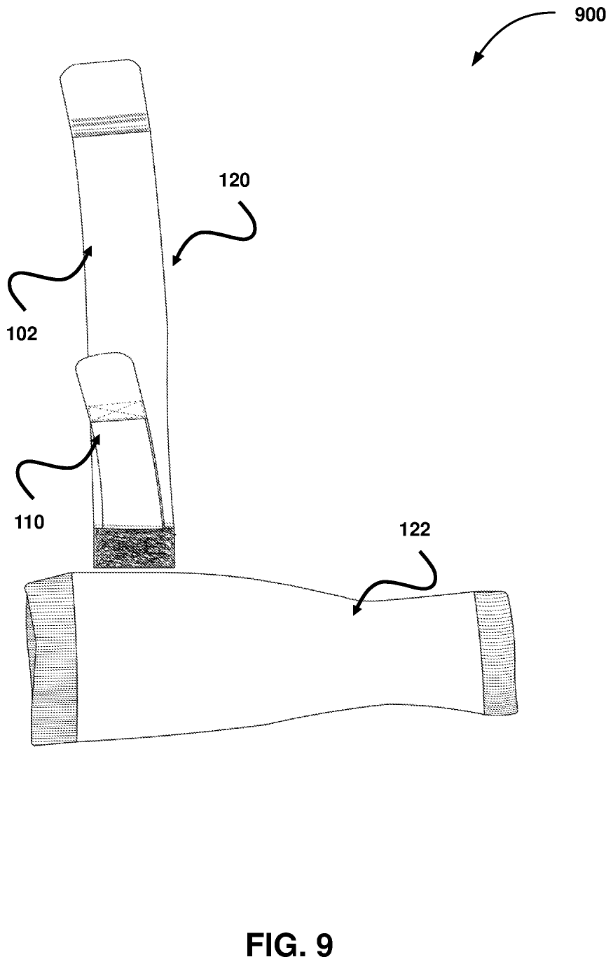

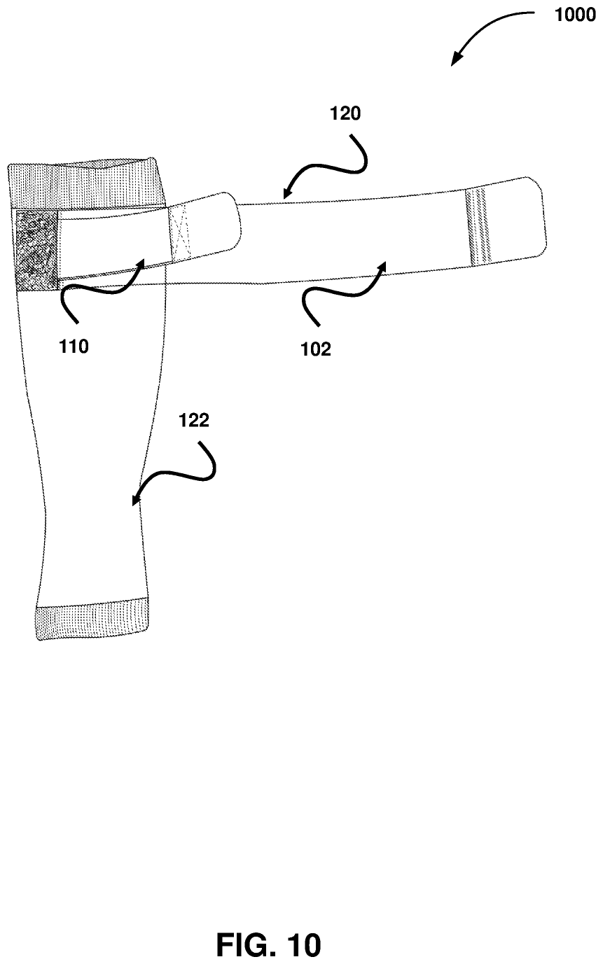

FIGS. 9 and 10 are diagrams 900 and 1000 that illustrate an attachment of the device i.e., a locking strap 120 to sleeve 122, in accordance with various embodiments of the present disclosure. To use the locking strap device 120, it needs to be attached to the sleeve 122 or clastic covering that goes around the targeted joint of a user. This attachment can be done by wrapping the locking strap 120 around the sleeve 122 and securing it in place using the Velcro hooks on the primary strap 102. The sleeve 122 should provide mild to moderate compression to the joint. The locking strap device 120 can be applied to various joints, such as the knee, ankle, or elbow, depending on the specific need. Once the locking strap device is attached to the sleeve, the user can wear it and adjust the secondary locking mechanism on top of the primary locking mechanism to fine-tune the pressure applied to the joint. The spiral pattern of pressure application provided by the device is designed to facilitate and lock the joint in place, reducing pain and providing support during physical activity or recovery.

The diagrams 900 and 1000 detail the practical application of the joint alignment device, particularly focusing on how the locking strap 120 integrates with the sleeve 122 to provide targeted support and therapeutic pressure to joints. This system is designed to offer a versatile solution for joint issues, adaptable to various parts of the body such as the knee, ankle, or elbow, catering to a wide range of needs and conditions. The locking strap device 120 is essentially a dynamic component of the overall device, functioning to secure the sleeve 122, which envelops the joint in place, while also allowing for the adjustment of pressure applied to the joint. The sleeve itself is designed to exert mild to moderate compression on the joint, aiding in stabilization and support without compromising comfort or circulation. This foundational support is crucial for ensuring that the therapeutic benefits of the device are maximized, providing a stable base upon which the locking strap can act. The method of attaching the locking strap 120 to the sleeve 122 is both straightforward and effective, utilizing Velcro hooks located on the primary strap 102. This design choice facilitates case of use, allowing users to easily wrap the locking strap around the sleeve and secure it in place with minimal effort. The presence of Velcro hooks not only ensures a secure attachment but also offers the flexibility to adjust the tightness and positioning of the strap as needed, accommodating different sizes and shapes of joints as well as varying levels of required support. Once the device is in place, the user has the capability to further adjust the level of pressure exerted on the joint through the secondary locking mechanism. This feature is particularly valuable for fine-tuning the device's fit and function, ensuring that the applied pressure is both therapeutic and comfortable. The spiral pattern of pressure application is a key aspect of the device's design, aimed at enhancing joint stability and mobility while also addressing pain and discomfort. This approach to pressure application is intended to mimic natural movement patterns and support the joint in a manner that facilitates healing and recovery, or simply supports ongoing physical activity. The adaptability of the locking strap device 120, in conjunction with the compressive sleeve 122, underscores the device's potential to provide significant relief and support for individuals dealing with joint pain, mobility issues, or in recovery from injury. The thoughtful integration of these components reflects a comprehensive approach to joint care, offering a non-invasive, customizable solution to enhance quality of life for those affected by joint-related challenges.

In an embodiment, the device 120 being described is a multi-step joint facilitation/locking system that is designed to apply pressure in a spiral pattern that is inherent in joints with an asymmetrical pattern. The device 120 is made up of a primary and secondary locking system, and elastics that can be stretched around a joint, such as the tibia, to provide support and facilitate joint movement. The primary locking mechanism has two stages. The initial stage involves a locking system that is held in place with a Velcro-like material. The second stage involves a joint facilitation tail that is not placed under as much tension as the initial stage and is also held in place with another Velcro-like material. This mechanism is designed to hold the lock in place and provide stability to the joint. The secondary locking mechanism is placed on top of the primary locking mechanism and is designed to allow for further joint facilitation and fine-tuning of how much force is applied to the joint. This provides the user with greater control and allows them to adjust the device as needed to suit their specific needs. The locking system is then attached to a sleeve or elastic covering that covers the length of the bone with mild to moderate compression. This compression is no more than 10-20 mmHg and allows the sleeve to be close to the skin. The sleeve also has a layer of silicone-like material underneath, which prevents the locking mechanism from sliding around on the skin. The device can be applied to various joints, such as the tibial-femoral joint (knee) via a spiral/lock pressure around the tibial, the tibial-fibular joint (ankle) via a spiral/lock pressure around the fibula, and the ulnar-humeral joint (elbow) via a spiral lock/pressure around the ulnar. Overall, this device provides users with a customizable and effective way to support and facilitate movement in various joints.

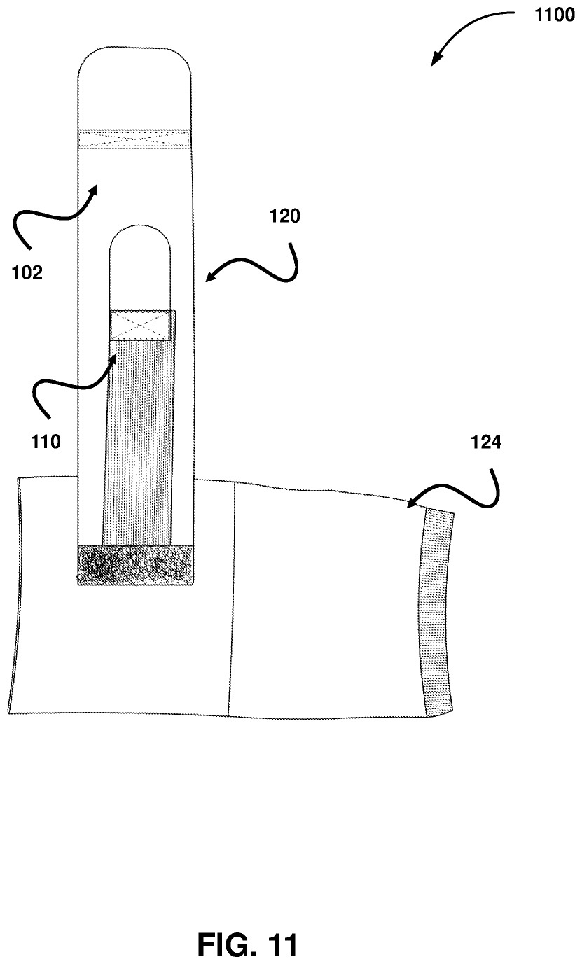

FIG. 11 is a diagram 1100 that illustrates an attachment of the device i.e., the locking strap 120 to a forearm sleeve 124, in accordance with various embodiments of the present disclosure. The locking device 120 includes the primary and secondary straps 102 and 110 that is attached (sewed) to the forearm sleeve 124. To attach the locking strap 120 to the forearm sleeve 124, the sleeve 124 is first slid over the forearm and positioned appropriately. The primary and secondary straps 102 and 110 are then wrapped around the sleeve and the joint that requires support. The anchor point on the secondary strap 110 is attached to the primary strap 102 using the cross-pattern stitching, and the Velcro locks on the primary strap 102 are fastened to secure the device in place. The locking strap system is designed to provide joint facilitation and locking in a spiral pattern, which is inherent in joints with an asymmetrical pattern. It helps to stabilize the joint and promote proper alignment, while also allowing for some movement and flexibility. The primary locking mechanism has a two-stage locking system, with the first stage lock being placed in the center of the primary strap and the second stage lock being placed at the end of the primary strap. This provides extra support and prevents the strap from slipping or shifting during use. The secondary locking mechanism is on top of the primary locking mechanism and allows for further joint facilitation and fine-tuning of how much force is applied to the joint. This can be particularly helpful in cases where the joint requires more or less support depending on the activity being performed. When attached to a forearm sleeve, the locking strap system can be used to provide support and stabilization for a variety of joint issues, including: (1) Tennis elbow—the device can be applied to the forearm to help reduce pain and inflammation in the tendons and muscles that attach to the elbow, (2) Golf elbow-similar to tennis elbow, the locking strap can help alleviate pain and inflammation in the forearm muscles and tendons, (3) Wrist injuries—the locking strap can provide support and stabilization to the wrist joint, helping to reduce pain and promote healing, (4) Carpal tunnel syndrome—the device can be used to support the wrist and reduce pressure on the median nerve, which can help alleviate symptoms of carpal tunnel syndrome, (5) Arthritis—the locking strap can be used to provide support and stabilization to joints affected by arthritis, helping to reduce pain and promote mobility, and the like applications. Overall, the locking strap system is a versatile and effective tool for supporting and stabilizing joints in the forearm and other parts of the body. It is particularly useful for individuals who engage in sports or other activities that place a lot of stress on their joints.

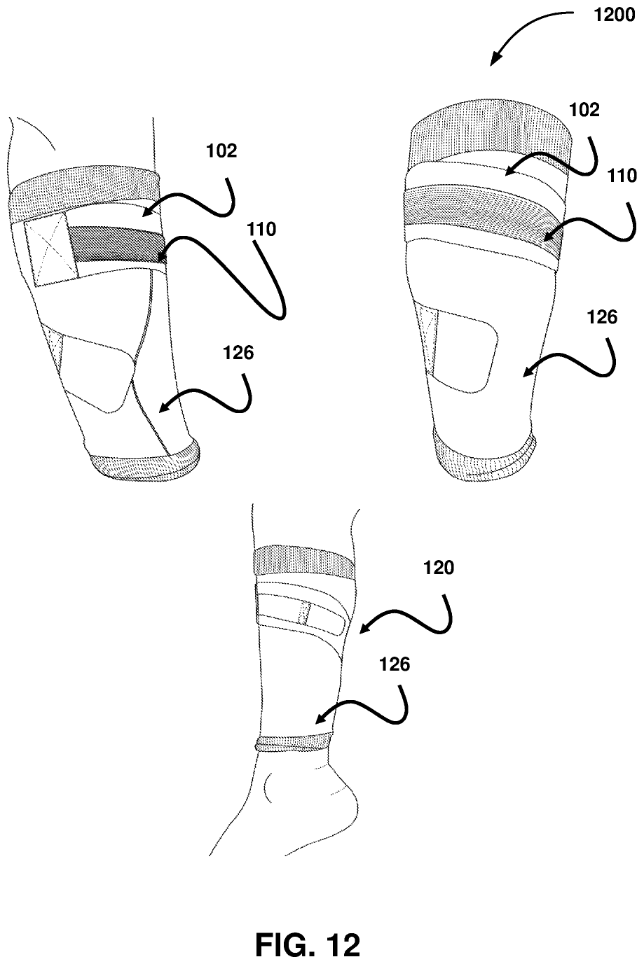

FIG. 12 is a diagram 1200 that illustrates an example of using the straps 102 and 110 on a leg, in accordance with various embodiments of the present disclosure. The locking device 120 includes the primary and secondary straps 102 and 110 that is attached (sewed) to a leg sleeve 126 that is worn by a user on his leg. The straps 102 and 110 attached to the leg sleeve 126 are typically used to provide additional support and stability to the knee joint. The leg sleeve is worn on the leg, and the straps are wrapped around the knee to provide compression and support. The primary strap 102 and secondary strap 110 are typically attached to the leg sleeve 126 using an anchor point on the sleeve. The straps 102 and 110 are then wrapped around the knee joint, with the primary strap 102 providing the primary level of support and the secondary strap 110 providing additional support and fine-tuning of the level of compression. The device is designed to apply pressure in a spiral pattern that is inherent in the joint with an asymmetrical pattern, such as the tibial-femoral joint (knee). The primary locking mechanism, which has a two-stage locking mechanism, is designed to hold the device in place using a Velcro-like material. The secondary locking mechanism is designed to provide additional joint facilitation and allow for fine-tuning of the amount of force applied to the joint.

The leg sleeve 126 is typically worn during physical activities that put stress on the knee joint, such as running, jumping, or playing sports. The compression provided by the leg sleeve 126 and straps 102 and 110 can help reduce pain and inflammation, improve blood flow, and provide additional support to the knee joint.

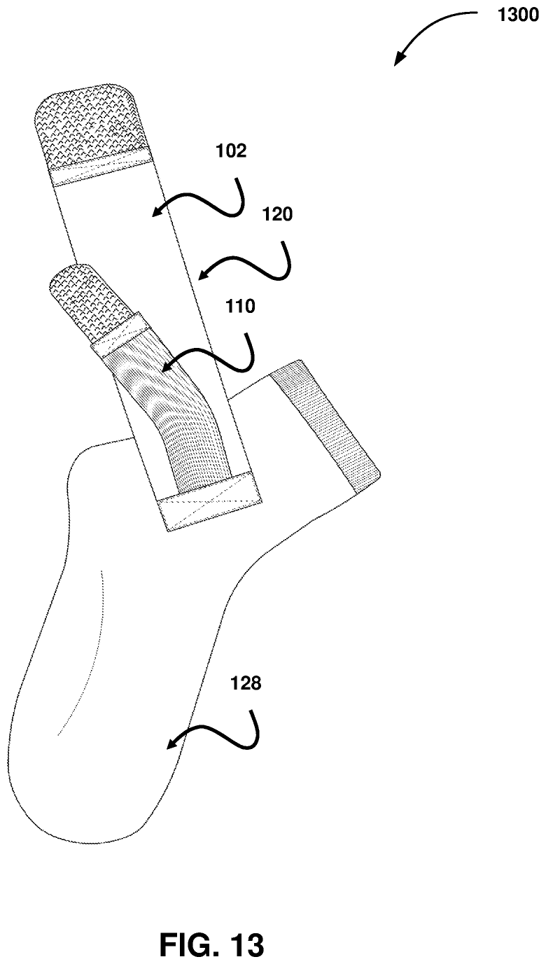

FIG. 13 is a diagram 1300 that illustrates an attachment of the device i.e., the locking strap 120 to a sock 128, in accordance with various embodiments of the present disclosure. The locking device 120 includes the primary and secondary straps 102 and 110 that is attached (sewed) to the sock 128. The locking device 120 can be attached to the sock 128 by sewing the primary strap 102 onto the sock 128. The secondary strap 110 can then be attached to the primary strap 102 using the anchor point and sewing it in place. The silicone side of the straps should be facing inward towards the user's skin. To wear and lock the device, the user would first put on the sock with the attached locking device. The device can then be wrapped around the joint, such as the ankle or knee, and the straps can be adjusted to the desired tension. The first stage lock can be secured in place by attaching the Velcro between the two ends of the primary strap. The second stage lock can then be applied to provide additional joint facilitation. The advantages of using a locking device attached to a sock include the ability to customize the fit and tension of the device to the user's needs. The sock provides a comfortable and secure base for the device, reducing the likelihood of slippage or movement during use. Additionally, the device can be easily removed and washed with the sock for convenience and hygiene purposes.

The Velcro fastening system is used on the primary and secondary straps 102 and 110 of the locking device 120 to provide a secure and adjustable fit around the joint. The Velcro consists of two components: the hook and the loop. The primary strap 102 has two locks such as Velcro lock. One lock is called as a first stage lock in which the Velcro is attached (sewed) between the two ends of the primary strap 102, for example in the centre. Another lock is called a second state lock in which the Velcro is attached (sewed) at the end of the primary strap 102. Similarly, the secondary strap 110 also has a Velcro attachment point. The Velcro hook component is sewn onto the strap, while the loop component is sewn onto the sleeve or sock. This allows the strap to be easily attached and detached from the sleeve or sock as needed. The Velcro also provides an adjustable fit, allowing the user to adjust the tension and compression around the joint for optimal support and comfort. Overall, the use of Velcro on the primary and secondary straps of the locking device provides a convenient and effective fastening system that allows for easy attachment and adjustment, enhancing the usability and effectiveness of the device.

The disclosed locking device 120 is designed to facilitate joint movement while providing locking support when necessary. The joint facilitation/locking system allows pressure to be applied in a spiral pattern that is inherent in joints with an asymmetrical pattern. This device is composed of a primary and secondary locking system, both of which are made up of elastics that can be stretched around a joint. The primary mechanism has a two-stage locking mechanism. The first stage of the primary locking system is an initial locking system that is held in place with a Velcro-like material. This locking system ensures that the elastic band is snugly fitted around the joint. The second stage of the primary locking system is a joint facilitation tail that is not placed on as much tension and is held in place with another Velcro-like material to hold the lock in place. This mechanism allows the user to fine-tune the force applied to the joint. The secondary locking mechanism is on top of the primary locking mechanism and allows for further joint facilitation and fine-tuning of how much force is applied to the joint. This secondary locking system is also composed of elastics that can be stretched around the joint. To wear the device, it is attached to a sleeve or clastic covering that covers the length of the bone with mild to moderate compression, typically between 10-20 mmHg. This tension allows the sleeve to be close to the skin and underneath the sleeve is a layer of silicone-like material to allow the locking mechanism to not slide around on the skin. This locking system can be applied to various joints, such as the tibial-femoral joint (knee), the tibial-fibular joint (ankle), and the ulnar-humeral joint (elbow). For example, for the tibial-femoral joint, the locking device is applied via a spiral/lock pressure around the tibial bone. The advantages of this device are that it allows for joint movement while providing support and locking when necessary, and it can be fine-tuned to apply the appropriate amount of force to the joint. The use of elastics and Velcro-like materials makes it easy to adjust and apply the locking system to different joints. The compression provided by the sleeve also helps with joint support and stabilization.

The primary support strap 102 is a two-stage locking mechanism that is designed to provide spiral pressure to a specific part of a joint, usually the main articulating bone distal to the joint structure. This primary strap 102 is composed of two parts—the initial lock and the facilitatory tail. The initial lock is located closer to the anchor point of the longer strap and is held in place by a Velcro-like material. It allows tension to be applied in a specific manner or direction to facilitate accessory joint motion. The facilitatory tail, which is not placed on as much tension as the initial lock, is held in place by another Velcro-like material to hold the lock in place. It allows the accessory motion to be moved while the bone moves into the primary motion. For example, in the Tibial Femoral joint (knee joint), the primary strap is placed on the lateral border of the tibial, which is the bone distal to the joint line. The first stage of the lock allows the medial rotation of the tibial, which restores the natural position of the tibial femoral joint. The second part of the elastic band is spiraled around the tibial, allowing the bone to move in a tri-planar motion as the knee moves through flexion and extension. The secondary lock 110 is located on top of the primary lock 102 and allows for additional pressure to be applied in the required direction, further assisting in promoting proper joint alignment. It also allows for fine-tuning of how much pressure is placed on the spiral movement. This elastic is silicone-lined to provide better grip and promote joint movement. The compression sleeve is designed to be a snug fit, allowing the locking mechanism to be as tight to the skin as possible. The sleeve covers the length of the bone with mild to moderate compression, no more than 10-20 mmhg, and is usually made of elastic material. Underneath the sleeve, there is a layer of silicone-like material to allow the locking mechanism to not slide around on the skin. The sleeve is essential in promoting joint movement and stability while the locking mechanism is in place.

In an alternate embodiment, overlapping locking mechanism may be incorporated into the described joint alignment device. This feature can be seamlessly integrated with the existing design, leveraging the primary and secondary straps' functionality while providing users with an even more customized fit and targeted pressure application. The overlapping locking mechanism may be added to the areas where the primary strap 102 and secondary strap 110 attach to the sleeve or sock (as described in diagrams 1200 and 1300), or directly onto the leg sleeve 126 and forearm sleeve 124 (as outlined in diagrams 1100 and 1200). This mechanism may involve additional segments on the straps that can overlap each other, enhancing the precision of pressure application. Each strap could feature extendable segments designed to overlap with corresponding segments on the same strap or the other strap. These segments can have Velcro or another type of fastener on their surfaces to securely lock in place once overlapped, offering a fine-tuning capability for the pressure applied around the joint. The anchor points on the secondary strap 110 may be designed to accommodate multiple positions of overlap, allowing users to customize the fit based on their specific needs and the particular joint's anatomy or condition. To ensure comfort and maintain the non-slip feature, the overlapping segments would also have a silicone layer on the side facing the skin, similar to the existing design of the straps. This mechanism allows for a more personalized fit, accommodating fluctuations in swelling or changes in joint stability over time. Overlapping segments can distribute pressure more evenly, reducing the risk of device slippage and improving therapeutic outcomes. It makes the device adaptable to a wider range of joint sizes and shapes, enhancing its utility across different body parts.

The foregoing descriptions of specific embodiments of the present technology have been presented for purposes of illustration and description. They are not intended to be exhaustive or to limit the present technology to the precise forms disclosed, and obviously many modifications and variations are possible considering the above teaching. The embodiments were chosen and described to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

While several possible embodiments of the invention have been described above and illustrated in some cases, it should be interpreted and understood as to have been presented only by way of illustration and example, but not by limitation. Thus, the breadth and scope of a preferred embodiment should not be limited by any of the above-described exemplary embodiments.

1. A joint facilitation and locking system comprising a primary and secondary locking mechanism, a compression sleeve, and elastics that can be stretched around a joint to apply pressure in a spiral pattern inherent in joints with an asymmetrical pattern, wherein the primary locking mechanism comprises a two-stage locking mechanism with an initial locking system and a joint facilitation tail held in place with a Velcro-like material, and the secondary locking mechanism allows for further joint facilitation and fine-tuning of the applied force.

2. The joint facilitation and locking system of claim 1, wherein the compression sleeve has a silicone-like layer to prevent the locking mechanism from sliding around on the skin.

3. The device according to claim 1, wherein the primary locking mechanism and the secondary locking mechanism are made of elastic material.

4. The device according to claim 1, wherein the compression sleeve has a compression range of 10-20 mmhg.

5. The device according to claim 1, wherein the silicone-like layer on the compression sleeve is made of medical grade silicone.

6. The device according to claim 1, wherein the joint facilitation tail of the primary locking mechanism is detachable and adjustable.

7. A joint alignment device comprising: a primary strap and a secondary strap each made of an elastic material and having a silicone side intended to face towards a user's skin to prevent slippage; an anchor point located on the secondary strap for attaching to the primary strap, where the primary strap includes a first stage lock and a second stage lock on its silicone side for securing the device in place; a locking strap configured to wrap around a sleeve or sock worn over a user's joint, where the sleeve or sock provides mild to moderate compression, and wherein the primary and secondary straps are designed to apply pressure in a spiral pattern around the joint for improved joint mobility and function; and a Velcro hook located on the non-silicone side of the straps for attaching the straps together, thereby enabling adjustment of the device's tension and compression around the joint.

8. The device of claim 7, wherein the first stage lock and the second stage lock are secured using Velcro material, allowing for adjustable tension and compression by the user.

9. The device of claim 7, further comprising a cross-pattern stitching at the anchor point to enhance the attachment strength between the primary and secondary straps.

10. The device of claim 7, wherein the sleeve or sock is selected from a group consisting of a leg sleeve, forearm sleeve, and a sock, each configured to support a specific joint including but not limited to the knee, ankle, elbow, and wrist.

11. The device of claim 7, wherein the silicone side of the straps features a textured surface to increase friction and prevent movement during physical activities.

12. The device of claim 7, wherein the locking strap includes an overlapping locking mechanism as an alternate embodiment, which comprises overlapping sections of the primary and secondary straps with additional Velcro fastening points to provide enhanced stability and customizable pressure distribution.

13. The device of claim 7, wherein the primary strap's first stage lock is located at the center between two ends of the strap, providing an initial secure fit around the joint.

14. The device of claim 7, wherein the second stage lock of the primary strap is located at one end of the strap, functioning as a joint facilitation tail to apply additional pressure in a spiral pattern.

15. The device of claim 7, wherein the secondary strap is attached on top of the primary strap, with its silicone side facing inward, towards the non-silicone side of the primary strap to enhance joint facilitation.

16. The device of claim 7, wherein the attachment of the primary and secondary straps to the sleeve or sock is performed by sewing, ensuring a durable and permanent attachment suitable for repeated use.

17. The device of claim 7, designed to address joint issues including but not limited to tennis elbow, golf elbow, wrist injuries, carpal tunnel syndrome, and arthritis by providing targeted pressure and support.

18. The device of claim 7, wherein the secondary strap's anchor point allows for fine-tuning of the force applied by the primary strap to the joint, enabling precise adjustment for optimal joint support.

19. The device of claim 7, wherein the Velcro fastening system of the primary and secondary straps provides a secure and adjustable fit, allowing for easy attachment and detachment from the sleeve or sock as needed.

20. The device of claim 7, wherein the device is configured to be easily removed and washed along with the sleeve or sock for hygiene and maintenance purposes, without compromising the integrity of the attachment points or the functionality of the locking mechanism.
